(19) United States
(12) Patent Application Publication
(10) Pub. No.: US 2010/0043792 A1
(43) Pub. Date: Feb. 25, 2010

(54) INHALER AND METHOD OF CONTROLLING THE SAME

(75) Inventor: Masahiro Takei, Kawasaki-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/508,841

(22) Filed: Jul. 24, 2009

(30) Foreign Application Priority Data
Aug. 20, 2008 (JP) 2008-211331

(51) Int. Cl. A61M 15/00

(52) U.S. Cl. 128/203.15

(57) ABSTRACT

To maintain an inner pressure of a tank within an appropriate range on a constant basis even when medicine is dispensed over plural times in multidose in a simple inhaler in which the inner pressure is not measured. A cartridge (10) includes an ejection head portion (1) for ejecting medicine to an air flow path of a inhaler and a reservoir (2). A piston member (122) for changing the inner volume of the reservoir (2) with the ejection from the ejection head portion (1) is connected to a piston shaft (61), which is a forwarding mechanism, by a connecting unit (117). The connection between the connecting unit (117) and the piston member (122) by an electromagnet (115) is released after the ejection from the ejection head portion (1), and the inner pressure of the reservoir (2) is balanced with the outside air pressure.

### BACKGROUND OF THE INVENTION

1. Field of the Invention

The present invention relates to a inhaler that is configured so as to be portably usable by a user and a control method therefor.

2. Description of the Related Art

An inhaler that ejects fine liquid droplets of medicine into an air flow path, through which air inhaled through a mouthpiece flows, by using an ejection principle of an ink-jet method, and allows a user to suction the ejected fine liquid droplets has been developed (refer to JP 2004-290593 A and JP 2004-283245 A). Such a inhaler has an advantage capable of spraying a predetermined amount of the medicine precisely by a uniformed particle size.

As basic components of such a inhaler includes an ejection head in which an ejection energy generating element such as a heater element is disposed, and a reservoir that contains the medicine supplied to the ejection head.

The inhaler capable of dispensing medicine over plural times by connecting to an ejection head while ensuring the capacity of a reservoir of an ejection amount for plural times is known. This device has a syringe type reservoir connected to the ejection head, and has a mechanism for appropriately supplying the medicinal solution of the ejection head from the reservoir to the ejection head when dispensing medicine.

In the inhaler adopting the ink-jet method, an appropriate negative pressure needs to be ensured inside the head orifice to start satisfactory ejection after the ejection head is filled with medicine. When performing ejection with the ejection head having an ejection port of 3 μm, the pressure inside the ejection head orifice can be maintained in a range of between −1 kPa to −5 kPa with the outside air pressure as a reference. The pressure inside the orifice is substantially equivalent to the inner pressure of the reservoir connected to the ejection head.

In the inhaler capable of dispensing medicine over plural times, the appropriate negative pressure needs to be continuously maintained in the subsequent ejection after the head is filled with medicinal solution at the beginning. When the ejection starts, the inner pressure increases in the negative pressure direction as the inner volume of the tank decreases by the medicine sprayed from the head. In order to maintain the inner pressure in the appropriate range, a piston and the like needs to be pushed out so as to decrease the volume of the tank according to the consumption speed of the medicinal solution.

However, according to the technology of the prior art, the negative pressure of the inner pressure of the tank changes in the direction of the positive pressure when the push-out amount (pressurizing amount) of the piston becomes larger than the head ejection amount due to clogging of the nozzle of the orifice, and the like. If the medicine is further continuously dispensed, the subsequent extra pressure accumulates in addition to the residual pressure of the previous time. Thus, the inner pressure of the tank eventually becomes a positive pressure, whereby the medicine is pushed out to the orifice surface and the surface becomes wet with the medicinal solution, resulting in that ejection failure occurs.

### SUMMARY OF THE INVENTION

The present invention aims to provide a inhaler capable of appropriately maintaining the inner pressure of the tank on a steady basis with a simple configuration that does not measure the inner pressure, and a method of controlling the same.

In order to solve the above-mentioned problem, a inhaler of the present invention comprises: an air flow path for enabling a user to inhale medicine; an ejection head for ejecting the medicine to the air flow path; a reservoir for storing medicine to be supplied to the ejection head; a piston for changing an inner volume of the reservoir; a piston driver for driving the piston; and a device for connecting the piston and the piston driver before ejection from the ejection head, and releasing the connection of the piston driver and the piston after the ejection.

The present invention provides a method of controlling a inhaler for enabling a user to inhale medicine ejected from an ejection head. The method comprises: ejecting the medicine from the ejection head to an air flow path; changing an inner volume of a reservoir for supplying the medicine to the ejection head according to an ejection amount from the ejection head in the ejecting; and balancing an inner pressure of the reservoir and an outside air pressure after the ejecting.

The inner pressure of the reservoir can be appropriately maintained with the simple configuration and the operation flow of intermittently releasing the piston drive. Thus, a portable inhaler of high performance and low price can be realized.

Further features of the present invention will become apparent from the following description of exemplary embodiments (with reference to the attached drawings), in which like reference characters designate the same or similar parts throughout the figures thereof.

### BRIEF DESCRIPTION OF THE DRAWINGS

FIG. 1 is a perspective view illustrating an outer appearance of a inhaler according to one embodiment of the present invention.

FIG. 2 is a perspective view illustrating a state in which an access cover is opened in the inhaler of FIG. 1.

FIG. 3 is a perspective view illustrating an outer appearance of a cartridge.

FIG. 4A is a cross-sectional view of the cartridge illustrating a state before an ejection head portion and a reservoir are connected.

FIG. 4B is a cross-sectional view of the cartridge illustrating a state after the ejection head portion and the reservoir are connected.

FIG. 5 is a perspective view of the interior of the device, with the protection cover for driving section of the device of FIG. 2 being removed.

FIG. 6 is a view in which the outer package is all removed so that the mechanism section inside the body can be easily seen.

FIG. 7A is an explanatory view illustrating a configuration of a connecting unit in a configuration using the connecting unit in place of a movable rubber plug joint of the device of FIG. 4A.

FIG. 7B is a block diagram illustrating a configuration of a control unit in the configuration using the connecting unit in place of the movable rubber plug joint of the device of FIG. 4A.

FIGS. 8A and 8B are conceptual views illustrating a normal ejection operation.

FIGS. 9A, 9B, 9C, 9D and 9E are views illustrating the operation for maintaining the inner pressure of the tank.

FIG. 10 is a flowchart illustrating the overall operation of the inhaler.

FIG. 11 is a flowchart illustrating an initial processing routine.

FIG. 12 is a flowchart illustrating an ejection routine.

FIG. 13 is a flowchart illustrating a subroutine for performing the connecting operation of the connecting unit.

FIG. 14 is a flowchart illustrating a subroutine for forwarding a piston by a predetermined step.

FIG. 15 is a flowchart illustrating a subroutine for releasing the connection and retreating the connecting unit.

### DESCRIPTION OF THE EMBODIMENTS

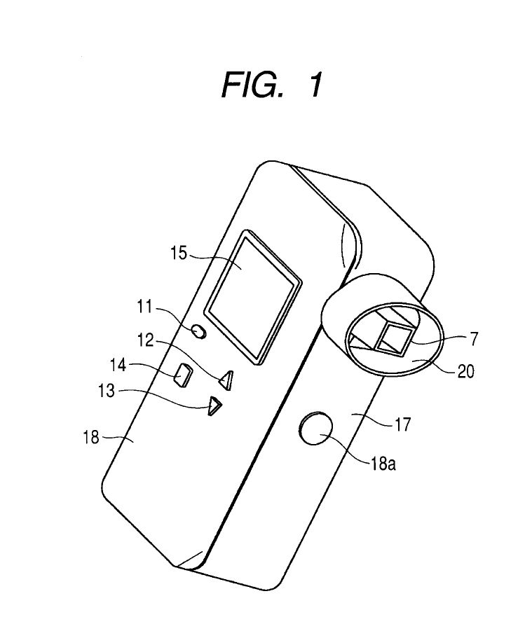

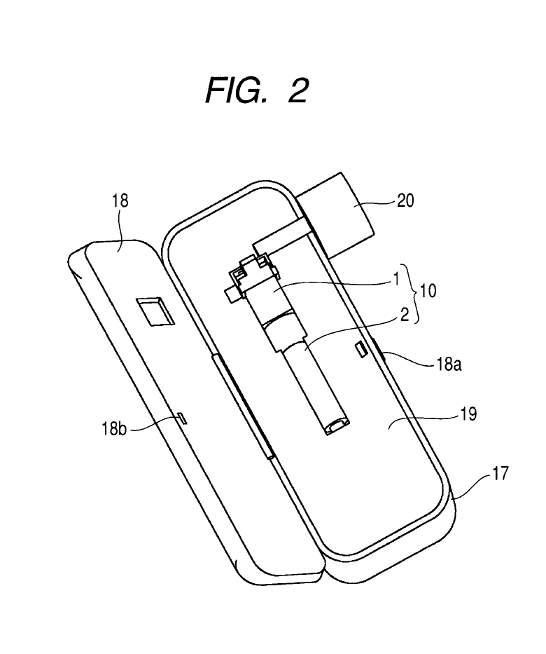

FIG. 1 is a perspective view illustrating an outer appearance of a inhaler according to one exemplary embodiment. A body outer package of the inhaler body is constituted by a housing case 17 and an access cover 18 which includes an unlock button 18a. As illustrated in FIG. 2, a hook 18b, which is provided such that the access cover 18 does not open during use, locks with a hook hooking shaft that operates integrally with the unlock button 18a biased by a spring. When opening the access cover 18, the hook is unhooked by pushing the unlock button 18a, and the access cover 18 opens by the force of a spring (not shown) biasing in the opening direction. The access cover 18 is provided with a display unit 15 for displaying a dosage, a time period, and an error, a menu switch button 11 for the user to perform the setting, an up button 12, a down button 13, and a select button 14 of a setting button.

FIG. 2 illustrates a state in which the access cover 18 is opened in the inhaler of FIG. 1. When the access cover 18 is opened, an ejection head portion 1 serving as a medicine ejection portion removable with respect to the device body, a reservoir 2 for storing the medicine to supply to the ejection head portion 1, and a protection cover 19 for driving section appear. The ejection head portion 1 ejects the medicine towards an air flow path 7. The user can inhale the medicine ejected into the air flow path by taking in air from a suction port (mouth piece) 20.

In this exemplary embodiment, the suction port 20 and the air flow path 7 are integrated. The suction port 20 may be disposed for every suction or may be cleaned and reused after the suction. The ejection head portion 1 and the reservoir 2 are replaced when the amount of medicine in the reservoir 2 becomes less than the amount of medicine to be dispensed in one suction. For instance, a function of counting the ejection amount may be provided to the body so that the remaining amount can be calculated by such ejection amount counting function, and thus the replacement timing can be notified to urge replacement to the user or ejection may not be performed until replacement is completed. The protection cover 19 for driving section is provided to prevent the user from easily touching the internal mechanism of the inhaler.

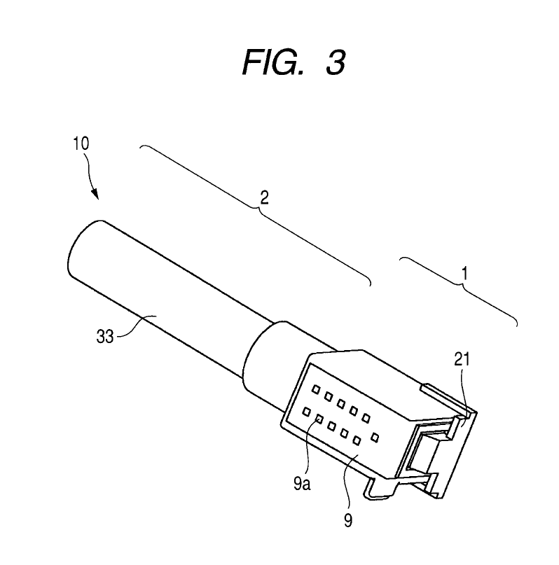

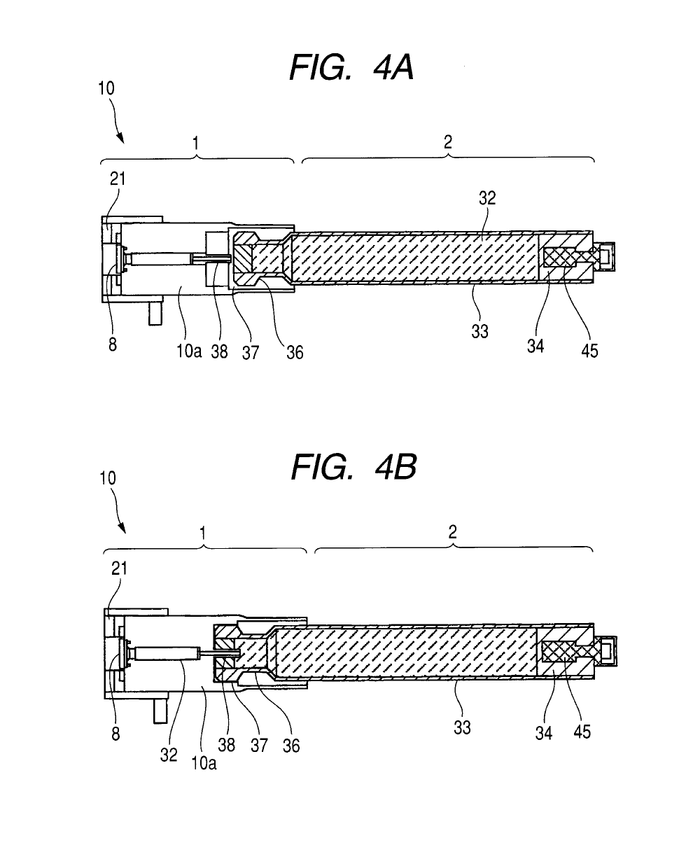

FIGS. 3, 4A, and 4B illustrate a configuration of the ejection head portion 1 and the reservoir 2.

FIG. 3 illustrates an outer appearance of a cartridge 10 including the ejection head portion 1 and the reservoir 2. FIGS. 4A and 4B illustrate the internal configuration of the cartridge 10, where FIG. 4A is a cross-sectional view illustrating a state before communicating the ejection head portion 1 and the reservoir 2 with each other, and FIG. 4B is a cross-sectional state illustrating a state after the communication.

The ejection head portion 1 includes an ejection head 8 arranged with a plurality of ejection ports, where the ejection head 8 is attached to and supported by a storing body 10a, and the medicine is supplied from the reservoir 2 to the ejection head 8.

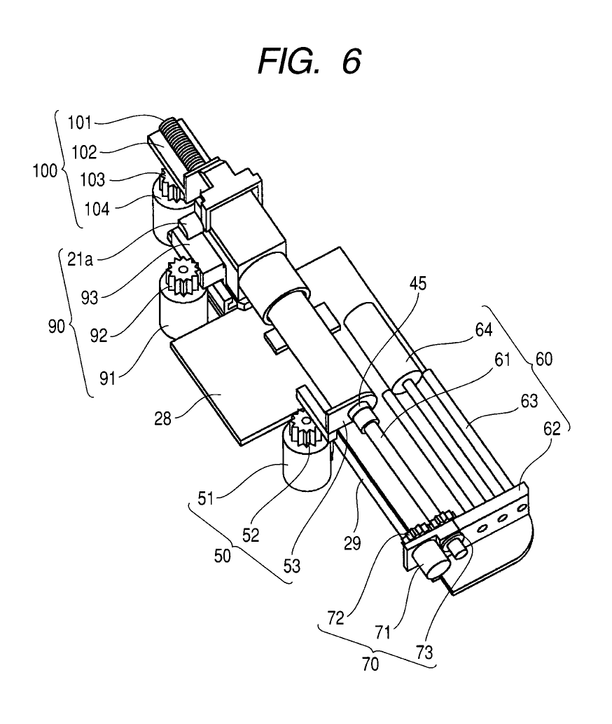

The ejection head 8 is provided with a heater serving as an ejection energy generating element near the ejection port, and hence the heated medicine is ejected from the ejection port with foam energy. An electrical wiring component 9 including an electrical contact 9a for supplying power to the heater is supplied with power from a chargeable battery 29 (see FIG. 6) serving as a secondary battery held in the inhaler body.

In order to protect the ejection head 8 from before being attached to the inhaler body, a head protection lever 21 including a medicine absorber is arranged so as to contact the ejection port surface of the ejection head 8. The head protection lever 21 is retreated in time of ejection so that the ejection port and the air flow path 7 communicate with each other.

The reservoir 2 includes a glass container (cylinder) 33 for storing medicine 32, where a fixed rubber plug 36 is held with an aluminum caulking fitting 37 at one end of the glass container 33. A movable rubber plug 34, which is a piston, for changing the inner volume of the container is fitted to the other end of the glass container 33 so as to shield the medicine 32 from outside air. As illustrated in FIG. 4A, the interior of the glass container is shielded from the outside air other than at the ejection port of the ejection head 8 at the stage the ejection head portion 1 and the reservoir 2 are connected to each other. The sealability of the reservoir 2 is maintained according to such configuration, thereby suppressing denaturation and change in concentration of the medicine 32 to a minimum.

As illustrated in FIG. 4B, when the reservoir 2 is pushed into the ejection head portion 1, a hollow needle 38 penetrates through the fixed rubber plug 36, thereby communicating the ejection head portion 1 and the reservoir 2 with each other. The medicine 32 is filled into the ejection head 8 by pushing-in the movable rubber plug 34, which is the piston. Thus, a piston driver for driving the piston of the reservoir 2 is connected to the movable rubber plug 34 by a movable rubber plug joint 45, which is a connecting unit.

The ejection head 1 and the reservoir 2 can be integrated to form the cartridge 10, and hence the user can easily insert the cartridge 10 to the inhaler body.

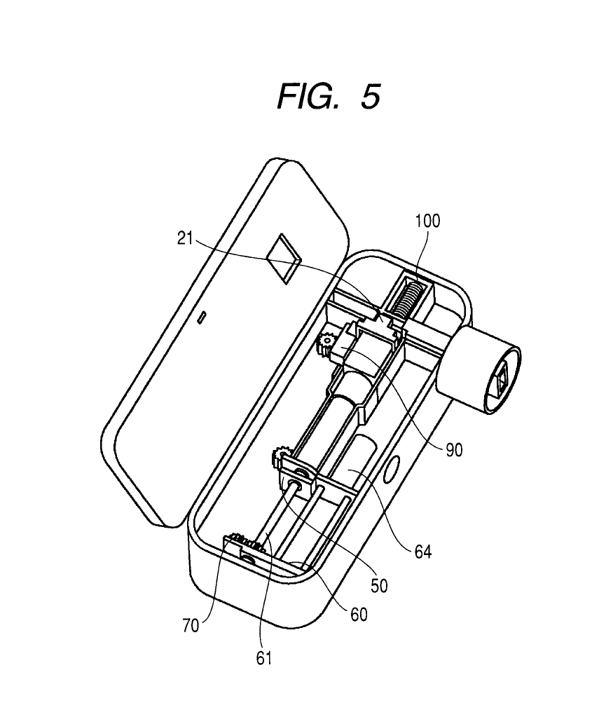

FIG. 5 is a perspective view of the interior of the device, with the protection cover 19 for driving section of FIG. 2 being removed.

First, a push-in unit 50 is provided for communicating the reservoir 2 and the ejection head portion 1 with each other so as to form a medicine path. A moving unit 60 for the movable rubber plug is arranged for moving the movable rubber plug 34 to the opposite side in the glass container 33, while the reservoir 2 is provided therebetween, so as to change the inner volume of the reservoir 2. The moving unit 60 for the movable rubber plug is a forwarding mechanism for drive-rotating a screw shaft motor 64 including a screw shaft to move a piston shaft 61. A piston shaft rotating unit 70 is arranged for performing the hooking operation of the movable rubber plug joint 45 and the piston shaft 61 so that the movable rubber plug joint 45 and the piston shaft 61 do not come out with the pulling operation. A head protection lever retreating unit 90 for moving the head protection lever 21 that is protecting the ejection head 8 and for opening the ejection surface is arranged on both sides of the cartridge 10. A head capping unit 100 for preventing drying of the ejection head 8 and attachment of dust in a state in which the ejection head 8 is attached to the body is provided on the upper side of the ejection head.

FIG. 6 is a view illustrating the mechanism section inside the body, with the outer package all being removed in FIG. 5. The push-in unit 50 is constituted by a motor 51 that generates a driving force for pushing in the reservoir 2 with respect to the ejection head portion 1, a pinion 52 press-fit to the motor shaft of the motor 51, and a push-in rack plate 53 partially having a rack shape that meshes with the pinion 52. The rack plate 53 is arranged to push the back end of the glass container 33.

Next, the moving unit 60 for the movable rubber plug serving as the piston driver is described. A piston shaft connecting plate 62 having a female screw, to which the screw shape of the screw of the screw shaft motor 64 provided at the motor main shaft conforms, is fitted with the screw shaft motor 64. A guide shaft 63 is arranged on both sides of the screw shaft to stop the rotation of the piston shaft connecting plate 62 and to guide the same in sliding. Thus, the rotational driving force of the screw shaft motor 64 slidably moves the piston shaft 61, and moves the rubber plug 34, which is the piston, connected by way of the movable rubber plug joint 45.

A piston shaft inverting unit 70 is incorporated in the piston shaft connecting plate 62. The piston shaft inverting unit 70 transmits the driving force of a piston shaft inversion motor 71 and an inversion motor gear 72 press-fit to the motor main shaft to a piston gear 73, and rotates the piston shaft 61. The rotation of the piston gear 73 engages the piston shaft 61 and the movable rubber plug joint 45 with each other, whereby the movable rubber plug 34 is pushed-in and pulled-out with respect to the glass container 33 thereby changing the inner volume so that the inner pressure is adjustable.

A control substrate 28 is arranged on the lower side of the cartridge 10. A CPU, a ROM, and a RAM serving as a drive control unit 4 for performing the body control such as control of each drive motor and varying of the ejection operation condition based on the measured pressure value are arranged on the control substrate 28.

A battery 29 serving as a driving source of each drive motor and an energy source for ejection is arranged on the lower side of the control substrate 28. Thus the inhaler can be easily used at any place because ejection and suction of the medicine are carried out with only the body.

The head protection lever retreating unit 90 is described. A pinion 92 press-fit to the main shaft of a motor 91 and a protection lever rack 93 having a rack shape mesh with each other. The protection lever rack 93 thus slidably moves and flips up a protruding portion 21a provided at the end of the head protection lever 21, rotating the head protection lever 21 and exposing the ejection head 8. The head protection lever retreating unit 90 is driven only when the cartridge 10 is inserted.

The head capping unit 100 is described. A capping plate 102 is slidable by way of a pinion 103 press-fit to the motor shaft by the driving force of a motor 104. The capping plate 102 slidably moves when meshed with the rack incorporated at the lower surface of the capping plate 102. The capping motor 104 is driven only when retreating the capping plate 102. The ejection head 8 is capped by the pressurizing force of a capping spring 101. This is because the ejection head 8 is to be capped even if the power of the body is turned OFF. That is, the head capping unit 100 is driven only when ejecting from the ejection head 8, and the ejection head is capped other than in time of ejecting medicine to prevent drying.

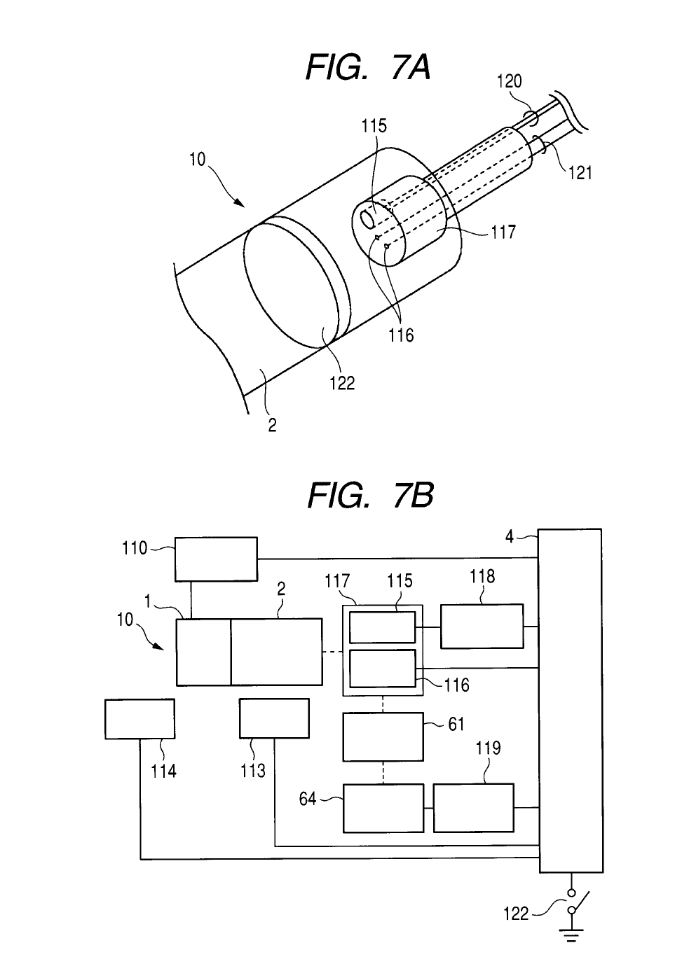

FIG. 7A illustrates a configuration using a connecting unit 117, which is a unit enabling connection and release of connection after ejection using an electromagnet 115 in place of the movable rubber plug joint 45 having a function of adjusting the inner pressure of the reservoir 2. A contact terminal 116 is buried in the connecting unit 117, and is installed such that conduction occurs when the connecting unit 117 contacts the end surface of the movable rubber plug 34. A piston member 122 made of a material such as iron, which has conductivity and is attractable to a magnet, is integrated to the end surface of the movable rubber plug 34. As illustrated in FIG. 7B, the contact terminal 116 is connected to the control unit 4 through a connecting line 121, and hence the contact can be confirmed by the control unit 4. The electromagnet 115 is attached to the end surface of the connecting unit 117, and is current-driven from an electromagnet driving circuit 118 through an electromagnet driving line 120.

A cartridge detecting sensor 113 for detecting that the cartridge 10 integrated with the head unit 1 and the reservoir 2 is inserted to the device is connected to the control unit 4. For the purpose of providing a head driving signal for spraying medicine, the head driving circuit 110 is connected to the ejection head portion 1 by way of the electrical contact 9a (see FIG. 3). The head driving circuit 110 is constituted by a gate array such as ASIC, and is designed to execute the desired spraying on its own by the control data and the activation signal from the control unit 4.

As illustrated in FIGS. 5 and 6, the piston shaft 61, which is the forwarding mechanism of the connecting unit 117, is driven by the screw shaft motor 64 to be hereinafter described. The screw shaft motor 64 is driven by the motor driving circuit 119 controlled by the control unit 4. The motor driving circuit 119 is configured by the gate array similarly to the head driving circuit 110.

The control unit 4 is also connected with a suction detecting sensor 114 for detecting that the patient has inhaled from the suction port 20. A power switch 112 for ultimately turning ON/OFF the power of the device is also connected.

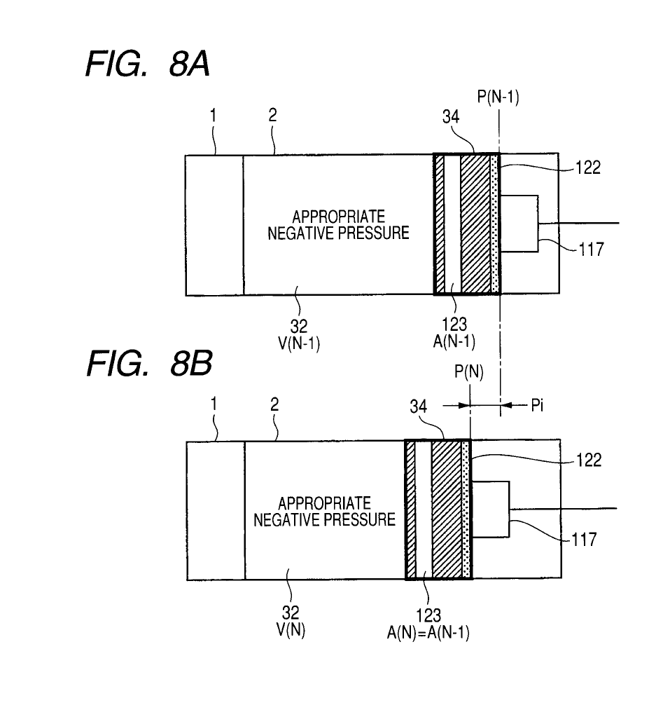

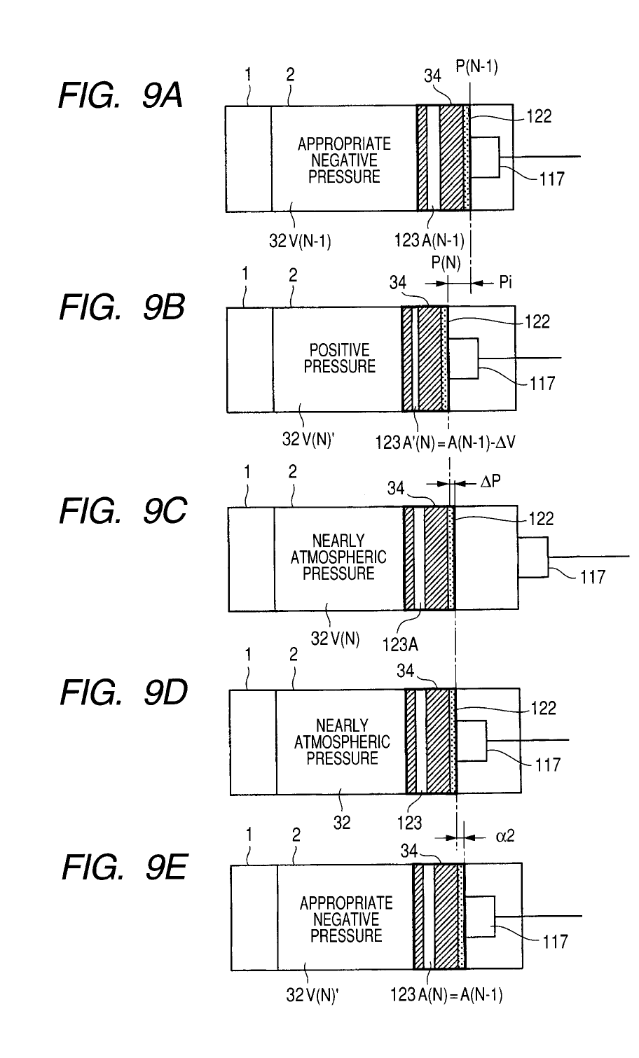

FIGS. 8A, 8B, and 9A to 9E are conceptual views describing the change in inner pressure of the reservoir 2 in the ejecting step of the ejection head 8 when the configuration of FIG. 7A is used.

FIGS. 8A and 8B schematically describe a case where the orifice is not clogged and the ejection amount and the rubber plug forwarding amount are equal, and FIGS. 9A to 9E schematically describe the operation of resolving the difference for a case where the ejection amount and the rubber plug forwarding amount are not equal to each other.

FIGS. 8A and 8B illustrate a state before and after one medicine ejecting operation, where FIG. 8A illustrates a state prepared to perform the Nₜₕ ejection after the (N−1)ₜₕ ejection. The inner pressure of the tank is ensured to an appropriate negative pressure by the operation flow described below. An air layer 123 existing at the distal end with respect to the advancing direction of the movable rubber plug 34 is illustrated as a model. The piston member 122 is a conductive plate to which the connecting unit 117 can connect, and is integrated with the movable rubber plug 34.

Here, V(N−1) is the volume of the medicine at the time, A(N−1) is the volume of the air layer 123 in the movable rubber plug, and P(N−1) is the rubber plug position and is the absolute position (number of pulse count CNT) from the starting point of the screw shaft motor 64. S(N−1) represents the inner pressure of the tank. FIG. 8B illustrates the state after the ejection for one time (ejection amount=Vi), where the numerical value changes in the following manner between before ejection and after ejection.

V(N−1)→V(N): V(N)=V(N−1)−Vi

Vi is the ejection amount (volume) per one time

A(N−1)→A(N): A(N)=A(N−1)

P(N−1)→P(N): P(N)=P(N−1)+Pi

Pi is the predetermined forwarding amount (number of pulses) per one time corresponding to Vi

S(N−1)→S(N)

Here, the state (volume) of the air layer 123 does not change because the inner pressure of the tank A(N−1) and A(N) are the same.

While FIG. 9A illustrates the same state as that of FIG. 8A, the state immediately after the ejection for one time is different. In FIG.9A, a case is supposed where the ejection amount Vi′ is reduced by ΔV from the normal ejection amount Vi due to clogging of the orifice. That is, as illustrated in FIG. 9B, the above-mentioned numerical values become as follows.

$${V ( N )}^{′} = V ( N - 1 ) - {Vi}^{′} \\ = V ( N - 1 ) - ( Vi - Δ V ) \\ = V ( N - 1 ) - Vi + Δ V \\ = V ( N ) + Δ V$$

A(N)′=A(N−1)−ΔV (ΔV represents a reduced amount of ejection amount) (volume))

P(N)=P(N−1)+Pi

S(N)>S(N−1)

Pi is set without taking into consideration the reduced amount of ejection amount, and thus corresponds to the number of steps of the motor drive for advancing a predetermined amount defined in advance, which is the same as that in the case of FIG. 8B.

After that, as illustrated in FIG. 9C, the connection of the connecting unit 117 is once released for the purpose of balancing the inner pressure and the outside air pressure with each other. Regarding the movable rubber plug 34, the cylinder inner diameter and the rubber plug diameter are selected so that the outside air pressure is balanced therewith in a very short period of time. ΔP is the rubber plug movement amount (number of pulses) after balanced. A is the volume of the air layer 123 when balanced with the outside air.

Subsequently, as illustrated in FIG. 9D, the connecting unit is again connected before the ejection to ensure the negative pressure for the next ejection. The rubber plug position P(N)′ (=CNT value) after the inner pressure is adjusted is represented as follows.

P(N)′=P(N−1)+Pi−ΔP

As illustrated in FIG. 9E, when the connecting unit is pulled back to ensure the negative pressure and the amount of being pulled back (number of steps) is represented by α2, the above-mentioned numerical values become as follows.

$${V ( N )}^{′} = V ( N - 1 ) - {Vi}^{′} \\ = V ( N - 1 ) - ( Vi - Δ V ) \\ = V ( N - 1 ) - Vi + Δ V \\ = V ( N ) + Δ V$$

$${A ( N )}^{′} = A ( N - 1 )$$

$${P ( N )}^{′} = P ( N - 1 ) + Pi - Δ P - α 2$$

S(N) is an appropriate negative pressure and is equal to S(N−1), and A(N)′ is the same as the value before the ejection.

If the operation is carried out in a sufficiently short period of time, problems do not arise in terms of operation even if the operation started after the ejection command is issued. Generally, when the movable rubber plug 34 is pushed according to the known predicted ejection amount calculated from the ejection condition, the pressure in the interior of the container becomes positive if the actual ejection amount is small due to nozzle clogging etc., and such state is maintained. If the movable rubber plug 34 is repeatedly pushed at less than the actual ejection amount to prevent the interior of the container from becoming a positive pressure, the negative pressure may gradually accumulate for every ejection. However, the inner pressure of the container can be reset to the level close to zero by releasing the connecting unit 117 from the movable rubber plug 34 after the ejection is terminated (every time or once every few times).

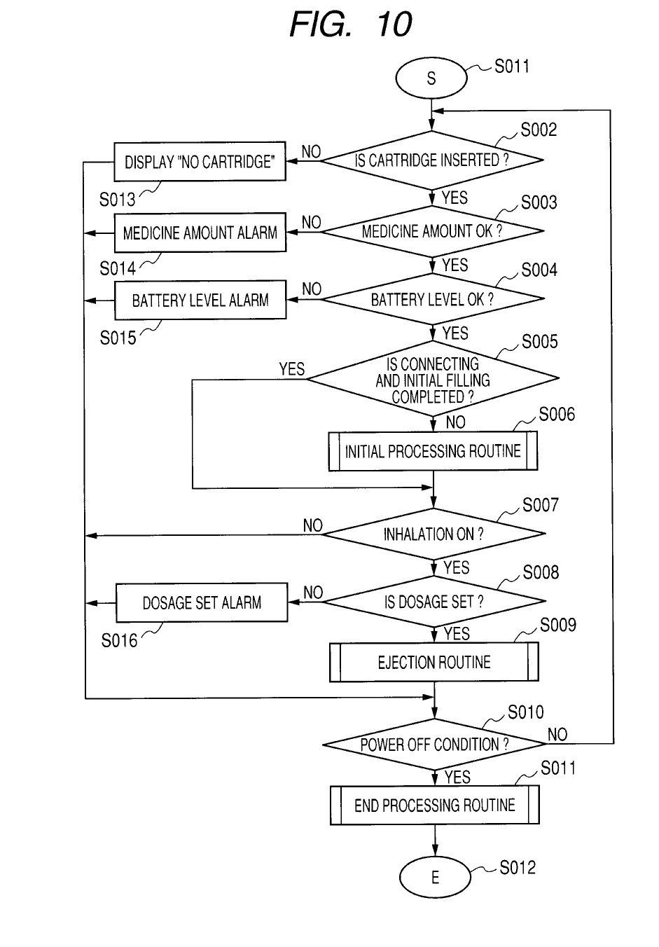

FIG. 10 illustrates the flowchart of the overall operation of the inhaler. The operation starts from step S001 when the power is turned ON by the power switch 112.

The state of the cartridge detecting sensor 113 is first detected to determine whether or not the cartridge 10 is inserted. The display of “no cartridge” is made (S013) if not inserted, and whether the medicine amount is sufficient is determined if inserted (S002). The medicine amount alarm is issued (S014) if the medicine amount is insufficient, and the battery level is determined if the medicine amount is sufficient (S003). The determination of the medicine amount may be made with software method. In other words, whether or not the liquid amount obtained by subtracting the total dosage of the medicine used in the past from the first amount of medicine is greater than the maximum ejection amount in the next ejection is determined. The battery level is determined if the medicine amount is sufficient in S003, the battery level alarm is issued (S015) if the battery level is insufficient, and the ejection routine after S005 starts if the battery level is sufficient (S004).

If the cartridge is newly inserted, the medicine filling process needs to be performed because the medicine is not yet filled in the ejection head portion 1. The determination on whether or not the detected cartridge is new can be made by recording the final information of the cartridge in a non-contact IC tag and reading the same. In another method, the piston position of the reservoir 2 can be optically read, and the determination can be made from the position information. In still another method, a claw member attached to one part of the cartridge storing body may be bent after the initial filling is completed.

When determined as the cartridge in which the initial filling has already been completed in the determination of S005, the initial processing routine S006 is skipped, and the suction timing waiting determination (S007) is made. In the initial processing routine S006, the medicine initial filling with respect to the head and the operation for ensuring the negative pressure to prepare for the next spraying are performed.

When the start of suction is detected in the determination of S007, whether or not the predetermined dosage necessary for spraying is already set is determined (S008). The dosage is set with the up button 12, the down button 13, and the select button 14 described in FIG. 1. If the dosage is fixed every time, the set value used in the past is used for the dosage. An alarm is issued to the user to set the dosage (S016) if the dosage is not set in the determination of S008, and the ejection routine (S009) starts if the dosage is already set.

After the ejection is completed in the ejection routine S009 to be hereinafter described, whether or not in power OFF condition is determined (S010). The condition for determining power OFF may be detecting that the patient operated the power switch 112, or the power may be turned OFF every time when dispensing of medicine is completed. The power OFF may be determined when the determination of low battery is made. The end processing routine starts (S011) when the power OFF condition is met in S010.

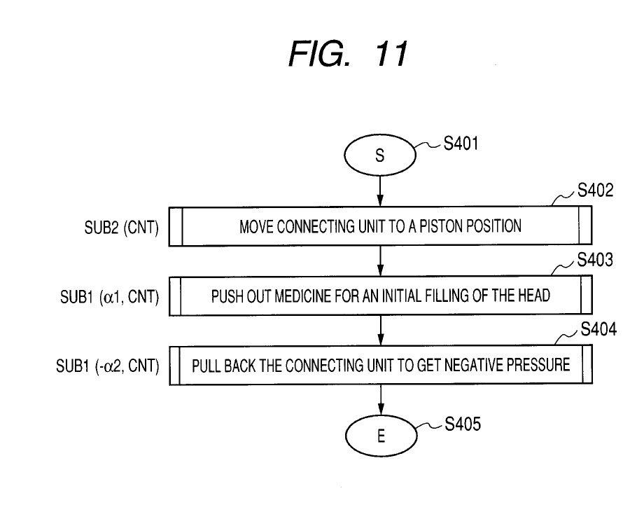

FIG. 11 illustrates a flowchart of the initial processing routine S006 of FIG. 10. After the routine starts from S401, a subroutine (SUB1) for moving the connecting unit 117 and connecting the connecting unit 117 to the piston member 122 is activated (S402). In this case, the connecting unit position (CNT) at the relevant time is input as an argument. In time of the initial filling, the connecting unit is retreated to the initial position and thus CNT=0. After the connection is completed in S402, a subroutine (SUB2) is activated to perform the push-out for head initial filling, and the medicine is filled to the head (S403). The subroutine (SUB2) is then activated to ensure the negative pressure (S404) in preparation for the initial ejection. Here, the connecting unit 117 is moved to the opposite side with respect to the interior of the container. The SUB2 drives the stepping motor by the desired number of steps, and provides the necessary number of steps (N) and the current connecting unit position CNT as arguments. The number of steps (α1) necessary for the head initial filling is used in S403, and the number of steps (−α2) necessary for ensuring the negative pressure is used in S404. The numerical value of such number of steps with which the operation is reliably guaranteed is determined in advance in the developing stage in view of the dimensional variation of the product and the insertion variation of the cartridge. According to experiments, the initial filling is found to be performed at 50 μL. For instance, for the case where the syringe inner diameter is 10.5 mm,

50 μL=Π*(10.5 mm/2)**2**d mm

The number of pulses necessary for the stepping motor to move 1 mm is number of pulses=161, and hence α1=91.

With respect to α2, the negative pressure that generates according to the remaining amount of the medicinal solution of the medicinal tank is not uniform even if the value is the same, and thus the α2 is changed in eight stages according to the following table. Similarly, if the medicinal solution tank diameter is 10.5 mm and the total medicinal solution volume is 1.5 mL, the total moving length is about 17 mm. Further, the forwarding number of pulse per 1 mm is 161, and hence the total forwarding number of pulse is 2700 pulses.

| Range of CNT value | α2 value |
|---|---|
| ~300 | 10 |
| 301~600 | 9 |
| 601~900 | 8 |
| 901~1200 | 7 |
| 1201~1500 | 6 |
| 1501~1800 | 5 |
| 1801~2100 | 4 |
| 2101~2400 | 3 |
| 2401~ | 2 |

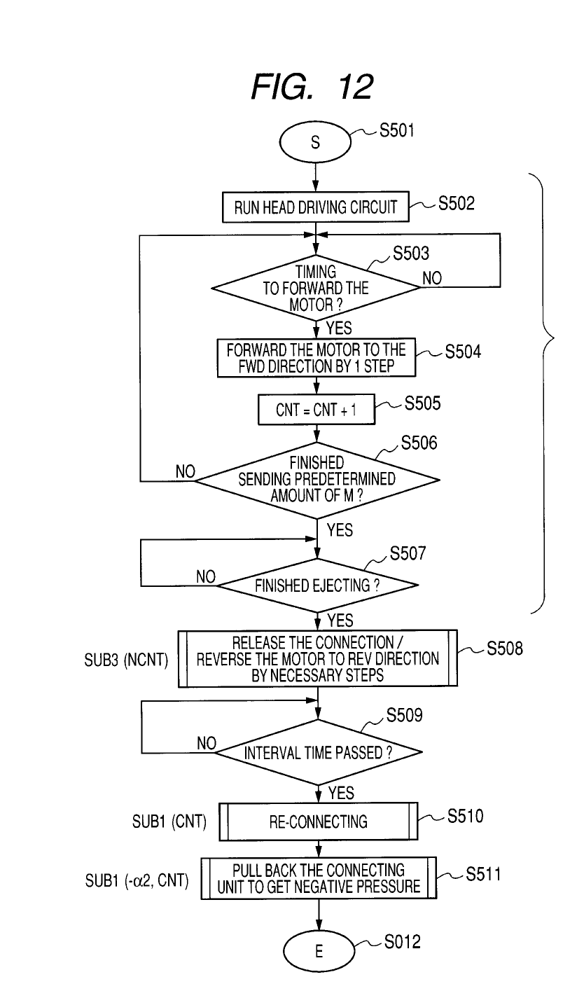

FIG. 12 illustrates a flowchart of the ejection routine S009 in FIG. 10. After activated in S501, the head driving circuit 110 is activated according to the head driving parameter from the control unit 4 (S502). Once activated, the head driving circuit 110 performs the head ejection operation independent from the flowchart described later. The medicine ejection volume per unit time is determined from the head driving parameter, and thus the screw shaft motor (piston motor) 64, which is the feeding motor of the connecting unit 117, needs to be synchronously driven and moved accordingly. This is to maintain the inner pressure of the tank within an appropriate range by matching the medicine volume per unit time ejected from the head and the forwarding amount of the connecting unit 117 during the ejection. The timing to forward the screw shaft motor 64 merely needs to be a time in which the ejection head ejects a volume corresponding to the push-out amount of the medicine for one pulse of the stepping motor (S503). After such time has elapsed, the piston motor is forwarded to the FWD direction by one step (S504), and the stepping motor position CNT is counted up by one (S505). The routine is repeated until such operation is performed for the necessary dosage of M times (S506). After that, the completion of the ejection operation in the head driving circuit 110 that is activated in S502 and independently operated is checked, and the routine is terminated if completed (S507). The stepping motor position CNT is returned as a variable.

A specific numerical value example is illustrated below. Here, calculation is made using the medicine syringe used in the experiment by way of example. When the syringe inner diameter is 10.5 mm and the ejection is carried out at 20 μL/second, 20 μL/second=Π*(10.5 mm/2)**2*d mm/second is met with the piston movement length per unit time as d mm/second, where d=0.227 mm/second. The number of pulses necessary for the stepping motor used in the experiment to move 1 mm is 161, which is 36.5 pulse/second. That is, the stepping motor needs to be moved 27 millisecond in time trade off per one pulse. If the dosage per one time is 50 μL, the total forwarding number of pulses M is 91.

The routine for providing a balance between the inner pressure of the tank and the outside air then starts. A routine for releasing the connection of the connecting unit 117 from the movable rubber plug 34, and reversing the motor by a necessary amount is activated (S508). In this case, the motor does not need to be retreated to the home position. This state is held until the timing at which the next ejection can be performed (S509). Meanwhile, the balance with the outside air of the movable rubber plug 34 is attained. After the necessary time has elapsed, a subroutine SUB1 for reconnecting the connecting unit 117 is activated (S510). After the connection is completed, a subroutine (SUB2) for pulling back the connecting unit by a predetermined number of steps α2 is activated so as to ensure the negative pressure for the next ejection (S511).

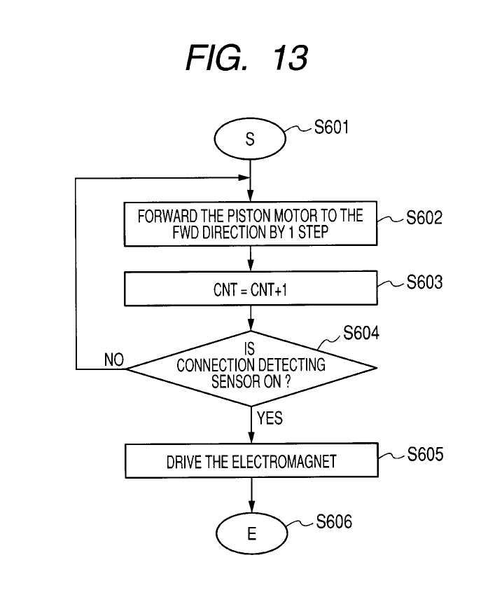

FIG. 13 illustrates a flowchart of the connecting subroutine SUB1. After activated in S601, the piston motor is forwarded to the positive direction FWD by one step (S602), and the stepping motor position CNT is counted up by one (S603). This operation is repeated until the connecting sensor 116 contacts thereto (S604) and the electromagnet is driven when detected (S605).

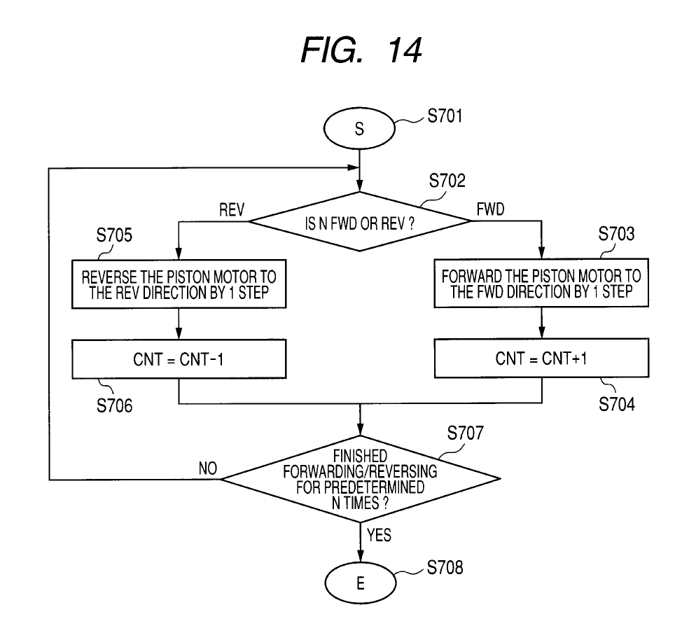

FIG. 14 illustrates a flowchart of the piston motor forwarding subroutine SUB2. After activated in S701, it is determined whether the direction is the positive direction FWD or the negative direction REV according to the specified argument N (S702). The argument N specifies a positive integer in the case of the positive direction, that is, the direction of forwarding the piston, and specifies a negative integer in the case of the negative direction, that is, the direction of pulling back the piston. In the case of the positive direction, the piston motor is forwarded to the positive direction by one step (S703) and the stepping motor position CNT is counted up by one (S704). In the case of the negative direction, the piston is pulled back to the negative direction by one step (S705), and the stepping motor position CNT is counted down by one (S706). The routine is terminated (S707) after such operation is repeated for a predetermined N times. The stepping motor position CNT is returned as a variable.

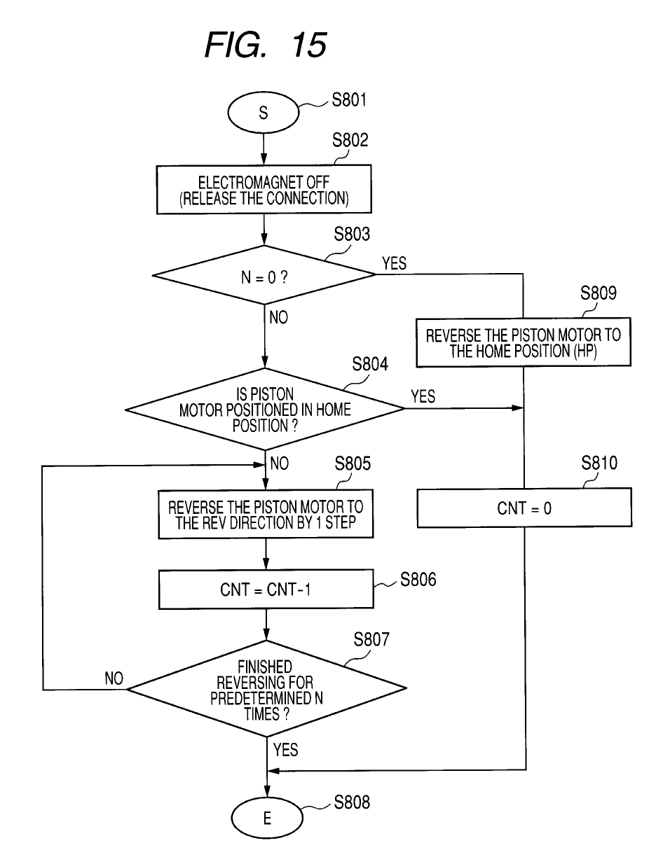

FIG. 15 illustrates a flowchart of the subroutine SUB3 for releasing the connecting unit and reversing the motor activated in FIG. 12. The argument is the necessary forwarding amount N and the current connecting unit position CNT. If N=0 is specified, the connecting unit 117 is moved back to the initial position (home position). If N≠0, the connecting unit 117 is returned by N number of steps. The specific forwarding amount of about 0.5 mm is sufficient in the above-mentioned experiment, in which case, N=80 steps.

After the routine starts in S801, the current drive of the electromagnet 115 is stopped (S802). Then, whether N=0 is determined (S803). If N≠0, the routine for forwarding by N times after S804 starts. If N=0, the motor is reversed to the home position (S809), the CNT=0 is reset (S810), and the routine is terminated. In S804, the routine is terminated through S810 if the piston motor is already at the home position (S804) and the motor is reversed by one step at a time while subtracting the CNT value if the piston motor is not at the home position (S805, S806). This operation is repeated until forwarding is completed for a predetermined N times (S807).

The inhaler of the present invention is usable for a variety of purposes besides that for inhaling the medicine. For example, the inhaler is usable as a spray ejection device of an air freshener or the like, a inhaler of a delicacy item such as nicotine, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-211331, filed Aug. 20, 2008, which is hereby incorporated by reference herein in its entirety.

What is claimed is:

1. A inhaler comprising:
an air flow path for enabling a user to inhale medicine;
an ejection head for ejecting the medicine to the air flow path;
a reservoir for storing medicine to be supplied to the ejection head;
a piston for changing an inner volume of the reservoir;
a piston driver for driving the piston; and
a device for connecting the piston and the piston driver before ejection from the ejection head, and releasing the connection of the piston driver and the piston after the ejection.

2. A inhaler according to claim 1, wherein the inner pressure of the reservoir is balanced with an outside air pressure by releasing the connection between the piston driver and the piston.

3. A method of controlling a inhaler for enabling a user to inhale medicine ejected from an ejection head; the method comprising:
ejecting the medicine from the ejection head to an air flow path;
changing an inner volume of a reservoir for supplying the medicine to the ejection head according to an ejection amount from the ejection head in the ejecting; and
balancing an inner pressure of the reservoir and an outside air pressure after the ejecting.
